(19) United States
(12) Patent Application Publication
(10) Pub. No.: US 2014/0276360 A1
(43) Pub. Date: Sep. 18, 2014

(54) ELECTROPHORECTIC DRUG COATED BALLOON AND CONDUCTIVE POLYMER COATING

(71) Applicant: ABBOTT CARDIOVASCULAR SYSTEMS INC., Santa Clara, CA (US)

(72) Inventors: Stephen D. Pacetti, San Jose, CA (US); Dariush Davalian, San Jose, CA (US); John Stankus, Campbell, CA (US); Mikael Trollsas, San Jose, CA (US); Syed Hossainy, Hayward, CA (US); Michael Ngo, San Jose, CA (US)

(21) Appl. No.: 13/843,082

(22) Filed: Mar. 15, 2013

(51) Int. Cl. A61N 1/30

(52) U.S. Cl. CPC A61N 1/306
USPC 604/21

(57) ABSTRACT

Balloon catheter configured to deliver a therapeutic agent upon provision of electric potential to the balloon, the catheter comprising an electrode disposed proximate the outer surface of the expandable member, a coating disposed on at least a portion of the outer surface, the coating including a therapeutic agent; and a power source in electrical communication with the electrode is described.

### FIELD OF THE INVENTION

The disclosed subject matter is related to the delivery of drugs from an insertable medical device. More particularly, the disclosed subject matter relates to a medical device including a balloon for delivery of a therapeutic agent, the balloon configured to release the therapeutic agent upon application of electric current.

### BACKGROUND OF THE INVENTION

Atherosclerosis is a syndrome affecting arterial blood vessels. It leads to a chronic inflammatory response in the walls of arteries, which is in large part due to the accumulation of lipid, macrophages, foam cells and the formation of plaque in the arterial wall. Atherosclerosis is commonly referred to as hardening of the arteries although the pathophysiology of the disease manifests itself with several different types of lesions ranging from fibrotic to lipid laden to calcific. Angioplasty is a vascular interventional technique involving mechanically widening an obstructed blood vessel, typically caused by atherosclerosis.

During angioplasty, a catheter having a tightly folded balloon is inserted into the vasculature of the patient and is passed to the narrowed location of the blood vessel at which point the balloon is inflated to a fixed size using an inflation fluid, typically a solution of angiographic contrast media. Percutaneous coronary intervention (PCI), commonly known as coronary angioplasty, is a therapeutic procedure to treat the stenotic coronary arteries of the heart, often found in coronary heart disease.

In contrast, peripheral angioplasty, commonly known as percutaneous transluminal angioplasty (PTA), refers to the use of mechanical widening of blood vessels other than the coronary arteries. PTA is most commonly used to treat narrowing of the arteries of the leg, especially, the iliac, external iliac, superficial femoral and popliteal arteries. PTA can also treat narrowing of veins and other blood vessels.

It was determined that following angioplasty, although a blood vessel would be successfully widened, sometimes the treated wall of the blood vessel experienced abrupt closure after balloon inflation or dilatation, due to acute recoil or spasm. Interventional cardiologists addressed this problem by stenting the blood vessel to prevent acute recoil and vasospasm. A stent is a device, typically a metal tube or scaffold, which was inserted into the blood vessel following angioplasty, in order to hold the blood vessel open.

While the advent of stents eliminated many of the complications of abrupt vessel closure after angioplasty procedures, within about six months of stenting, a re-narrowing of the blood vessel can form, which is a condition known as restenosis. Restenosis was discovered to be a response to the injury of the angioplasty procedure and is characterized by a growth of smooth muscle cells—analogous to a scar forming over an injury. As a solution, drug eluting stents were developed to address the reoccurrence of the narrowing of blood vessels. One example of a drug eluting stent is a metal stent that has been coated with a drug that is known to interfere with the process of restenosis. A potential drawback of certain drug eluting stents is known as late stent thrombosis, which is an event in which blood clots form inside the stent.

Drug coated balloons are believed to be a viable alternative to drug eluting stents in the treatment of atherosclerosis. In a study which evaluated restenosis, and the rate of major adverse cardiac events such as heart attack, bypass, repeat stenosis, or death in patients treated with drug coated balloons and drug eluting stents, the patients treated with drug coated balloons experienced only 3.7 percent restenosis and 4.8% MACE as compared to patients treated with drug eluting stents, in which restenosis was 20.8 percent and 22.0 percent MACE rate. (See, PEPCAD II study, Rotenburg, Germany).

Although drug coated balloons are a viable alternative and in some cases may have greater efficacy than drug eluting stents as suggested by the PEPCAD II study, drug coated balloons present challenges due to the very short period of contact between the drug coated balloon surface and the blood vessel wall. The drug delivery time period for a drug coated balloon differs from that of a controlled release drug eluting stent, which is typically weeks to months. In particular for the coronary arteries, the balloon may only be inflated for less than one minute, and is often inflated for only thirty seconds. Therefore, an efficacious, therapeutic amount of drug must be transferred to the vessel wall within a thirty-second to one-minute time period. For the peripheral vasculature, the allowable inflation times can be greater than one minute, but are still measured in minutes. Thus, there are challenges specific to drug delivery via a drug coated balloon because of the necessity of a short inflation time, and therefore time for drug or coating transfer—a challenge not presented by a drug eluting stent, which remains in the patient's vasculature once implanted.

Various embodiments of drug-coated balloons have been proposed to address these needs, including balloons with a therapeutic agent disposed directly on the balloon surface and balloons having various protective sheaths. However, not all embodiments result in an efficacious response in reducing restenosis after balloon and/or bare metal stent trauma.

Therefore, a need exists for a drug delivery balloon, and more particularly, a balloon coated with a therapeutic agent that provides for effective delivery of the therapeutic agent from the surface of the balloon.

### SUMMARY OF THE INVENTION

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

In accordance with an aspect of the disclosed subject matter, a catheter for intraluminal delivery of a therapeutic agent to a subject is provided. The catheter includes an elongate shaft having a proximal end portion, a distal end portion, and an inflation lumen defined therebetween. The catheter further includes an expandable member coupled at the distal end portion of the elongate shaft, the expandable member having a proximal end, a distal end, an interior chamber defined therein, and an outer surface. The expandable member further includes an electrode disposed proximate the outer surface of the expandable member and a coating disposed on at least a portion of the outer surface, the coating including a therapeutic agent. The catheter additionally includes a power source in electrical communication with the electrode. Upon balloon inflation at the site of drug delivery, electric current or voltage can be supplied from the power source to the balloon electrode to provide an electromotive force to the therapeutic agent or charged moieties encapsulating the therapeutic agent, thereby effecting rapid and specific delivery of the therapeutic agent to the site of delivery.

In some embodiments of the disclosed subject matter, the electrode is a film of conductive material. In further embodiments, the conductive material is a metal material. In additional embodiments, the metal material is selected from the group consisting of gold, platinum, platinum iridium, silver, palladium, tantalum and niobium.

In additional embodiments, the conductive material of the electrode is a conductive polymeric material. In some embodiments, the conductive polymeric material is disposed on the balloon surface as a coating. In additional embodiments, the polymer coating includes carbon particles and/or metallic particles to improve conductivity of the coating.

In some embodiments according to the subject matter described herein, the therapeutic agent has no net electrical charge. In certain embodiments, the therapeutic agent is a cytostatic drug. In some embodiments, the cytostatic drug is selected from the group consisting of rapamycin, sirolimus, zotarolimus, everolimus, tacrolimus, and biolimus. In some embodiments, the therapeutic agent is zotarolimus.

In certain embodiments, a charged moiety such as a micelle, nanoparticle or liposome is employed to encapsulate the therapeutic agent. Where the therapeutic agent is neutral (i.e. possesses no net electrical charge), the unencapsulated therapeutic agent would otherwise not be subject to a net electromotive force in the presence of an electric field. In some embodiments, the therapeutic agent is disposed in an emulsifier. In some embodiments, the emulsifier is a surfactant. In still further embodiments, the surfactant is anionic.

Suitable anionic surfactants for use with certain embodiments of the disclosed subject matter include phosphatidylglycerols, phosphatic acids, lysophospholipids, and saturated and unsaturated fatty acids. In some embodiments, the surfactant is a phosphatidylglycerol selected from the group consisting of egg phosphatidylglycerol (EPG), dimyristoyl-phosphatidylglycerol (DMPG), palmitoly-oleoyl phosphatidylglycerol (POPG), and 1,2-distearoyl-sn-glycerol-3-phosphoglycerol sodium salt (DSPG). In additional embodiments, the surfactant is a phosphatidic acid selected from the group consisting of dimyristoyl-phosphatidic acid (DMPA), dipalmitoyl-phosphatidic acid (DPPA), and 1,2-distearoyl-sn-glycero-3-phosphatidic acid (DSPA). In still further embodiments, the surfactant is a lysophospholipid selected from the group consisting of lysophosphatidic acid (LPA), lyso-phosphatidylcholine (LPC), and sphingosine-1-phosphate (S1P).

In further embodiments, the therapeutic agent is encapsulated with a liposome comprising an anionic fatty acid. In some embodiments, the neutral therapeutic agent is encapsulated in a nanosphere comprising an anionic fatty acid. In additional embodiments, the neutral therapeutic agent is encapsulated in a microsphere comprising an anionic fatty acid. Suitable anionic fatty acids include phospholipids selected from the group consisting of phosphatidylethanolamine, purified 90% soya phosphatidylcholine (trade name LECIVA-S90), and purified egg lecithin (trade name LIPOVA-E120).

As disclosed previously, in certain embodiments, the balloon electrode is a conductive polymer coating. Suitable conductive polymers include polypyrrole, polyacetylene derivatives, poly(phenyl sulfide), polythiopene, and poly(3,4-ethylenedioxythiopene). In some embodiments, the conductive polymer is disposed as nanoparticles in the coating. In further embodiments, the conductive polymer is combined as a composite with an additional conductive polymer. In some embodiments, the conductive polymer is disposed as nanoparticles and combined as a composite with an additional conductive polymer. Suitable conductive polymers for combination as a composite include poly(vinylidene fluoride), poly(vinylidene fluoride-co-hexafluoropropylene), poly(ester-amide), and polyesters.

In some embodiments, the conductive polymer coating comprises a conductive polymer and a therapeutic agent disposed in a matrix configuration. The coating is configured to absorb ions and water upon balloon inflation and temporary application of an electric field from the external power source, leading to swelling of the coating. Upon swelling of the coating, the therapeutic agent elutes from the coating to the vessel lumen. In some embodiments, upon cessation of the electric field, the coating releases the fluids and reprises its initial configuration.

In some embodiments in accordance with the disclosed subject matter, the catheter balloon includes a surface coating comprising a conductive polymer that is piezoelectric. In these embodiments, the catheter does not necessarily comprise an external power source and an electrode in electric communication with the power source. Suitable piezoelectric conductive polymers include poled poly(vinylidene fluoride) and poled poly(vinylidene-trifluoroethylene). In some embodiments, the piezoelectric coating further comprises graphitic carbon.

In additional embodiments, the therapeutic agent is encapsulated in a complex that dissolves in electric current. Suitable complexes that dissolve in response to electric current include of gold, silver, porous gold nanoparticles, porous silver nanoparticles, gold-coated poly(vinylidene fluoride) nanoparticles, and silver-coated poly(vinylidene fluoride) nanoparticles.

In some embodiments of the disclosed subject matter, the therapeutic agent is selected from the class of antithrombotics, anticoagulants, antiplatelet agents, anti-lipid agents, thrombolytics, antiproliferatives, anti-inflammatories, agents that inhibit hyperplasia, smooth muscle cell inhibitors, antibiotics, growth factor inhibitors, cell adhesion inhibitors, cytostatic agents, cell adhesion promoters, antimitotics, antifibrins, antioxidants, antineoplastics, agents that promote endothelial cell recovery, antiallergic substances, viral vectors, nucleic acids, monoclonal antibodies, antisense compounds, oligonucleotides, cell permeation enhancers, radiopaque agent markers, HMG CoA reductase inhibitors, pro-drugs and combinations thereof.

In some embodiments, the balloon coating further comprises a plasticizer. Suitable plasticizers include, without limitation, glycerin, polyethylene glycol, and polypropylene glycol propylene glycol, polysorbates, N-methylpyrrolidone, dimethyl sulfoxide, benzyl benzoate, ethyl benzoate, benzyl alcohol, and phenoxyethanol. In some embodiments, the plasticizer increases the elongation capacity of the coating to maintain coating integrity during balloon inflation and deflation.

In some embodiments, the power source is a direct current power source external to the body of the subject. In some embodiments, the power source includes a timer. In additional embodiments, the power source includes a fast acting fuse. In some embodiments, the power source is connected to the electrode by an insulated electrical lead. In still further embodiments, the electrical lead engages the electrode at the proximal end of the expandable member.

It is to be understood that both the foregoing description and the following detailed description are exemplary and are intended to provide further explanation of the disclosed subject matter claimed.

The accompanying drawings, which are incorporated and constitute part of this specification, are included to illustrate and provide a further understanding of the systems of the disclosed subject matter. Together with the description, the drawings serve to explain the principles of the disclosed subject matter. The exemplified embodiments of the disclosed subject matter are not intended to limit the scope of the claims.

### BRIEF DESCRIPTION OF THE DRAWINGS

The disclosed subject matter will now be described in conjunction with the accompanying drawings in which:

FIG. 1A is a schematic view of one representative balloon catheter in accordance with the disclosed subject matter. FIG. 1B is a schematic cross-sectional end view taken along lines A-A in FIG. 1A. FIG. 1C is a schematic cross-sectional end view taken along lines B-B in FIG. 1A.

FIG. 2 is a schematic view of a system in accordance with the disclosed subject matter, including a representative balloon catheter and an electrode in electrical communication with an external power source.

FIG. 3 is a schematic representation of a system in accordance with the disclosed subject matter, with the balloon catheter positioned in a lumen of a blood vessel and an external electrode applied to the skin of the patient to complete an electric circuit to permit temporary application of a current to the balloon.

FIG. 4 is an illustration of electropolymerization of pyrrole into polypyrrole to form a conductive polymer.

### DETAILED DESCRIPTION

In accordance with an aspect of the disclosed subject matter, a catheter for intraluminal delivery of a therapeutic agent to a subject is provided. The catheter includes an elongate shaft having a proximal end portion, a distal end portion, and an inflation lumen defined therebetween. The catheter further includes an expandable member coupled at the distal end portion of the elongate shaft, the expandable member having a proximal end, a distal end, an outer surface, and an interior chamber defined therein. The expandable member further includes an electrode disposed proximate the outer surface of the expandable member and a coating disposed on at least a portion of the outer surface, the coating including a therapeutic agent. The catheter additionally includes a power source in electrical communication with the electrode. Upon balloon inflation at the site of drug delivery, voltage can be supplied from the power source to the balloon electrode to provide an electromotive force to the therapeutic agent or to charged moieties encapsulating the therapeutic agent, thereby effecting rapid and specific delivery of the therapeutic agent to the site of delivery. In accordance with a further aspect of the disclosed subject matter, the electrode can be a conductive polymer which reversibly attracts water in response to voltage supplied from the power source. Additionally or alternatively, the expandable member is coated with a piezoelectric coating and the therapeutic agent is encapsulated in particles which dissolve upon application of electric current.

Reference will now be made in detail to the various aspects of the disclosed subject matter. The method of the disclosed subject matter will be described in conjunction with the detailed description of the system, the figures and examples provided herein.

Unless otherwise defined, all technical and scientific terms used herein have the same meanings as commonly understood by one of ordinary skill in the art to which the disclosed subject matter belongs. Although methods and materials similar or equivalent to those described herein can be used in its practice, suitable methods and materials are described below.

It is to be noted that the term “a” entity or “an” entity refers to one or more of that entity. As such, the terms “a”, “an”, “one or more”, and “at least one” can be used interchangeably herein. The terms “comprising,” “including,” and “having” can also be used interchangeably. In addition, the terms “amount” and “level” are also interchangeable and can be used to describe a concentration or a specific quantity. Furthermore, the term “selected from the group consisting of” refers to one or more members of the group in the list that follows, including mixtures (i.e. combinations) of two or more members.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to +/−20%, or up to +/−10%, or up to +/−5%, or up to +/−1% of a given value. Alternatively, particularly with respect to biological systems or processes, the term can mean within an order of magnitude, or within 5-fold, or within 2-fold, of a value. With reference to pharmaceutical compositions, the term “about” refers to a range that is acceptable for quality control standards of a product approved by regulatory authorities.

The systems and methods presented can be used for delivery of a therapeutic agent to a vessel wall of a subject. The methods and systems presented herein can also be used for manufacture and assembly of medical devices such as a drug coated balloon catheter. While the disclosed subject matter references application of a therapeutic agent, it is to be understood that a variety of coatings including polymeric, therapeutic, or matrix coatings, can be applied to various surfaces of medical devices, as so desired.

Referring to FIG. 1, for purposes of illustration and not limitation, an exemplary embodiment of balloon catheter device in accordance with the disclosed subject matter is shown schematically in FIGS. 1A and 1B. As depicted in FIGS. 1A and 1B, the balloon catheter device 10 generally includes an elongated catheter shaft 12 having a proximal end and having a distal end and an expandable member or balloon 30 located proximate to the distal end of the catheter shaft. In accordance with the disclosed subject matter, an electrode 50 is applied to at least a portion of the working length of the balloon catheter. The expandable balloon has an outer surface and an inner surface disposed at the distal end portion of the catheter shaft.

For purpose of illustration and not limitation, an elongated catheter shaft 12 having a coaxial arrangement is shown comprising an outer tubular member 14 and an inner tubular member 16. The outer tubular member 14 defines an inflation lumen 20 disposed between the proximal end portion and the distal end portion of the catheter shaft 12. For example, and as illustrated in FIG. 1B, the coaxial relationship between the inner tubular member 16 and the outer tubular member 14 defines an annular inflation lumen 20. The expandable member 30 is in fluid communication with the inflation lumen 20. The inflation lumen therebetween can supply fluid under pressure to the expandable member 30, and establish negative pressure to draw fluid from the expandable member 30. The expandable member 30 can thus be inflated and deflated. The elongated catheter is sized and configured for delivery through a tortuous anatomy, and can further include a guidewire lumen 22 that permits it to be delivered over a guidewire 18. As illustrated in FIG. 1B, the inner tubular member 16 defines the guidewire lumen 22 for the guidewire 18. Although FIGS. 1A and 1B illustrate the guidewire lumen as having an over-the-wire (OTW) construction, the guidewire lumen can be configured as a rapid-exchange (RX) construction, as is well known in the art. Similarly, the shaft can be provided as a multilumen member, or composition of two or more tubular members, as is known in the art.

As further depicted in FIG. 1A, the expandable member or balloon 30 has a distal end 32, a proximal end 34 and a working length “L” therebetween. The expandable member embodied herein has a an interior chamber 36 in fluid communication with the inflatable lumen 20 of the elongated shaft 12. Any of a number of suitable expandable member constructions and shapes can be used, as described further below.

In accordance with the disclosed subject matter, at least one therapeutic agent 40 is disposed along at least a portion of the working length “L” of the expandable member 30. The at least a portion of the working length can be a selected length of the working length or the working length in its entirety. Furthermore, the at least a portion can reference a pattern on the surface of the working length, such as rings, dots, linear or curvilinear segments, or another design. The at least one therapeutic agent can be disposed along the portion of the working length of the expandable member in any suitable manner that will allow for release from the expandable member to the vessel wall. For example, the at least one therapeutic agent can be applied as a coating to the outer surface of the expandable member. Additionally or alternatively, the expandable member can be provided with reservoirs or similar surface features to contain therapeutic agent for release therefrom. Furthermore, pores or channels can be defined along a portion of the working length for infusion-type release of the therapeutic agent therefrom. The at least one therapeutic agent can be disposed alone, e.g., neat, or in combination with a suitable additive, such as a surfactant, plasticizer or the like. Additionally, and as described further below, the at least one therapeutic agent can be disposed for delivery over an electrode 50. For example, the therapeutic agent 40 can be applied as a layer over the electrode, and/or the therapeutic agent can be mixed with or encapsulated in further coating components as appropriate.

Additionally, and as depicted schematically in FIG. 1, an electrode 50 can be provided to provide an electromotive force to the coating and/or therapeutic agent upon application of current or voltage from a power source. The electrode 50 can be an anode or a cathode, as described further below. Suitable electrodes include without limitation metallic or conductive polymer films, as described further below.

Conventional drug-coated balloons rely on a combination of mechanical compression of the drug against the vessel wall and passive diffusion of the drug from the balloon coating following balloon inflation to transfer the drug to the site of delivery. The disclosed subject matter, in contrast, further provides an electromotive force to promote rapid and specific drug release from the balloon coating to the vessel lumen and/or a user-controlled system to initiate and hasten drug release from the balloon coating to the vessel lumen by providing and/or generating voltage or current.

In accordance with the subject matter disclosed herein, the catheter balloon includes an electrode disposed proximate to the surface of the balloon. The electrode is in electric communication with a power supply. The coating comprising the therapeutic agent of the system disclosed herein can be disposed over the surface of the electrode or can form the electrode itself. Upon delivery of the catheter balloon to a vessel lumen and inflation of the balloon, electric current is temporarily provided from a power source to the electrode. The electric current provides an electromotive force which repels electrostatically charged molecules and/or moieties from the balloon to the vessel wall.

In some embodiments, and as illustrated in FIG. 2, electrical communication between the balloon electrode 50 and the power supply 60 is established by an insulated electrical lead 70 provided inside the catheter. The lead 70 extends along the catheter to a point proximate to the proximal end of the balloon. As depicted herein, for the purpose of illustration and not limitation, the lead 70 is attached to the external surface of the balloon taper in contact with the balloon electrode 50. The therapeutic agent 40, shown mixed into or encapsulated in a coating, is disposed over the balloon electrode 50.

As shown in FIG. 3, an electrical circuit is formed, such as by providing an opposite electrode placed on the body of the subject. Such electrodes are common in medical practice, such as EKG electrodes, which can be affixed to the patient with a conductive gel layer. Upon intraluminal delivery and inflation of catheter balloon device 10, power is provided from the power source 60 for about 30 seconds to about 60 seconds. Additionally, the power source 60 can include a timer and/or a fast-acting fuse to prevent undesired electrical circuits within the body of the subject.

As illustrated in FIG. 3, the power source can be an external power source. Additionally or alternatively, the power source can be integrated as a component of the catheter system. Furthermore, the integrated power source can be battery powered. For example, the battery of the integrated power source can be replaceable or can be disposable.

The balloon electrode can be provided in a variety of forms. For example, the balloon electrode can be a film disposed on all or a part of the working surface of the balloon. Additionally or alternatively, the electrode can be formed within the balloon wall or within the interior of the balloon. The electrode can be provided in any desired shape or pattern on the balloon surface. The electrode can comprise a conductive metal, including, for example and not limitation, platinum, platinum iridium, silver, tantalum, niobium, palladium, or gold. The conductive film or layer of the electrode is disposed on the balloon surface by, for example, sputtering, metal evaporation, electroless plating, or mechanical adherence of the electrode material. In certain embodiments, a metallic electrode layer is provided on a low compliance balloon to preserve mechanical integrity of the metallic electrode layer upon balloon inflation. Additionally or alternatively, the electrode can comprise a conductive polymeric coating, such as a polymer capable of expanding when inflated. The conductivity of the polymer coating can be increased by the inclusion of suitable carbon or metallic particles. Suitable conductive polymeric coatings are provided below.

Electric potential is provided from the power source to the electrode to impart an electric field to the balloon. This electric field will exert an electrostatic force on charged molecules in the vicinity of the field. In certain embodiments, the therapeutic agent itself can be provided as a charged molecule. Alternatively, the therapeutic agent can be encapsulated in a charged moiety, such as a micelle.

For example, if the therapeutic agent is positively charged or encapsulated in a positively charged moiety, the balloon electrode can be configured as an anode to provide the desired electromotive force. Alternatively, if the therapeutic agent is negatively charged or encapsulated in a negatively charged moiety, the balloon electrode can be configured as a cathode to provide the desired effect.

In accordance with alternative embodiments, the electric field can be generated by delivery of voltage to the balloon electrode. For example and not limitation, the voltage can be between about 10 millivolts and about 5 volts; or the voltage can be between about 100 millivolts and about 2 volts; or the voltage can be between about 1 volt and about 2 volts.

In certain embodiments according to the disclosed subject matter, the therapeutic agent is a cytostatic drug, including, for example, zotarolimus, sirolimus, rapamycin, everolimus, biolimus, umirolimus, myolimus, novolimus, temsirolimus, deforolimus, ridaforolimus, tacrolimus, pimecrolimus, and combinations thereof. Alternatively, the therapeutic agent can be an anti-proliferative drug, including for example, paclitaxel, protaxel, docetaxel and combinations thereof. Such cytostatic and anti-proliferative drugs can have a net neutral charge, and can be encapsulated in a charged moiety to permit electrophoretic delivery. Furthermore, by using a highly hydrophobic drug, it is possible to readily encapsulate the therapeutic agent in surfactant micelles, microspheres, liposomes, or nanoparticles. Suitable negatively-charged surfactants for micellar encapsulation according to the disclosed subject matter include, for example, phospholipids, such as phosphatidylglycerols, phosphatidic acids, lysophospholipids, and fatty acids. Suitable positively charged surfactant encapsulants include positively charged sorbitan esters, polysorbates, and poloxamers. Depending on the desired stability of the encapsulant, low critical micelle concentration (CMC) surfactants can be selected to produce stable micelles, or high CMC surfactants can be selected to produce relatively less stable micelles.

Suitable phosphatidylglycerols include, without limitation, egg phosphatidylglycerol (EPG), dimyristoyl-phosphatidylglycerol (DMPG), palmitoly-oleoyl phosphatidylglycerol (POPG), and 1,2-distearoyl-sn-glycerol-3-phosphoglycerol sodium salt (DSPG). Smaller phosphatidylglycerols, including DMPA, are particularly suitable for the formation of micelles encapsulating hydrophobic drugs. Suitable phosphatidic acids include, for example, dimyristoyl-phosphatidic acid (DMPA), dipalmitoyl-phosphatidic acid (DPPA), and 1,2-distearoyl-sn-glycero-3-phosphatidic acid (DSPA). Suitable lysophospholipids include, for example, sphingosine-1-phosphate and lysophosphatidic acid. Lysophospholipids contain a single fatty acid chain, which have relatively large polar head groups in comparison to single acyl side chains, and therefore are especially suited to micelle formation. With respect to fatty acids, both saturated and unsaturated fatty acids are suitable for micellar encapsulation of hydrophobic agents. Suitable anionic fatty acids include, without limitation, phosphatidylserine, phosphatidylinositol 4,5-bisphosphate and phosphatidylinositol 3,4,5-triphosphate.

Additionally or alternatively, a hydrophobic therapeutic agent, such as a cytostatic or cytotoxic drug, can be solubilized in a positively or negatively charged polymer. Upon application of current or voltage to the balloon electrode, the resulting electromotive force will propel the polymer and therapeutic agent solution from the balloon to the vessel wall. Suitable positively charged polymers include, without limitation, poly(vinylbenzyl trialkyl ammonium), poly(4-vinyl-N-alkyl-pyridiumiun), and poly(acryloyl-trialkyl ammonium), as well as positively charged polysaccharides, such as cellulose, dextran and starch. Suitable negatively charged polymers include, without limitation, carboxymethyl cellulose, sodium carboxymethyl cellulose, carboxymethyl celluiose-cysteine, poly(acrylic acid), poly(methacrylic acid), poly(L-aspartic acid), poly(D-aspartic acid), poly(L-aspartic acid) sodium salt, poly(L-glutamic acid), poly(D-glutamic acid), and poly(L-glutamic acid) sodium salt. Polyionic polymers exhibit significant tissue adhesion, and can in some embodiments promote adhesion and retention of the coating and therapeutic agent after delivery from the balloon.

Electrophoresis generally will occur more rapidly with smaller moieties having greater diffusivity. Accordingly, micelles, liposomes and nanoparticles generally will electrophorese more rapidly than microspheres. Therapeutic agent-encapsulating moieties, including nanoparticles, can be formed by dispersing or sonicating an organic solution of the therapeutic agent and the selected encapsulant. The therapeutic agent-encapsulating moieties can then applied over the balloon electrode by dipping, spraying, or by other techniques known in the art. One technique particularly suited to the application of positively-charged moieties is disclosed in U.S. Pat. No. 8,298,607, incorporated herein by reference in its entirety.

In accordance with another aspect of the disclosed subject matter, the balloon electrode comprises a conductive polymer disposed on the surface of the catheter balloon. The conductive polymer can further be doped with suitable dopants as desired to permit oxidation or reduction of the conductive polymer. Depending on the chemistry and doping of the conductive polymer and doping, upon application of voltage to the conductive polymer, the conductive polymer becomes oxidized and temporarily attracts ions and aqueous fluid. For purpose of example, and not limitation, a polypyrrole conductive polymer doped with an anion can be reversibly oxidized upon provision of voltage from the power source to the conductive polymer. In its oxidized state, the conductive polymer will attract ions and aqueous fluid. In its reduced state, the conductive polymer will expel ions and water from the bloodstream and/or tissue into the coating. Where the conductive polymer is disposed as a coating or a coating comprising the therapeutic agent is disposed over the conductive polymer, hydration of the coating promotes swelling and release of the coating comprising the therapeutic agent. By subsequently ceasing provision of voltage or reversing the voltage in the circuit, the conductive polymer returns to its reduced state and/or is oxidized to release the water and ions to the surrounding tissue by diffusion. This cycle of oxidation and reduction to attract and release solvent can be repeated several times during balloon deployment as desired.

In accordance with another aspect of the disclosed subject matter, drug delivery can be achieved by both the electromotive force that results from application of voltage to the conductive polymer and the reversible reduction (i.e. hydration) of the coating polymer. For the purpose of illustration and not limitation, the coating can include charged surfactant particles encapsulating a hydrophobic drug as set forth above. Upon balloon inflation and the application of voltage from the power source to the conductive polymer, the resulting electromotive force will repel the charged encapsulants from the surface of the balloon into the tissue. Concomitantly, the oxidation of the conductive polymer will promote the flow of ions and water into the balloon coating, resulting in hydration and swelling of the coating. Coating swelling will permit more rapid diffusion of the charged surfactant encapsulants to the vessel wall.

In accordance with the above, as well as additional embodiments, the conductive polymer itself can be loaded with drug in a matrix format at up to 50 percent therapeutic agent by weight. Upon application of voltage to the conductive polymer, the polymer will hydrate and swell, permitting elution of the drug. Additionally, by reversing the direction of voltage from the power supply, the flow of water and ions into the coating is reversed, permitting diffusion of the drug from the polymer coating to the vessel wall.

Conductive polymers suitable for the disclosed subject matter generally can be characterized by alternating single and double bonds along the polymer chain. Such conductive polymers for use as the balloon electrode include, without limitation, polypyrrole, polyacetylene derivatives, poly(phenylene sulfide), polythiopene, and poly(3,4-ethylenedioxythiopene). Additionally or alternatively, the conductive polymers listed above can be provided as nanoparticles and composited with an additional conductive polymer such as poly(vinylidene fluoride), poly(ester-amide) or a polyester to augment coating conductivity. Additionally or alternatively, the therapeutic agent itself can be disposed as nanoparticles in the conductive polymer.

In some embodiments, the conductive coating is loaded with the therapeutic agent in a matrix format, as known to those in the art. The therapeutic agent can be disposed without an encapsulant. Additionally, the therapeutic agent can be encapsulated, such as within microspheres, or by encapsulating nanoparticles or surfactant liposomes, microspheres as disclosed above.

In certain embodiments, the conductive polymer is polypyrrole. As illustrated in FIG. 4, the coating of conductive polymer can be formed by electrochemical oxidation of pyrrole on an anode surface, such as the surface of the balloon itself or a separate surface. If a separate surface is employed, the polypyrrole coating is subsequently applied to the balloon surface using suitable techniques known in the art.

According to still another aspect of the disclosed subject matter, the conductive coating can include a piezoelectric property. In such embodiments, cyclical inflation and deflation of the balloon itself can be configured to generate a local electrical current. This intrinsically supplied current can, in certain embodiments, dissolve susceptible coatings to release a therapeutic agent. Furthermore, the various aspects described above can be employed without the need for an external power source in electrical communication with the balloon.

In certain embodiments, the piezoelectric coating includes a therapeutic agent encapsulated in porous nanoparticles of a colloidable metal, such as gold or silver. Additionally or alternatively, the therapeutic agent-encapsulating nanoparticles can be included with the piezoelectric polymer. Upon generation of voltage by the piezoelectric effect, the nanoparticles encapsulating the therapeutic agent can dissolve for release of the therapeutic agent. Repeated cycles of balloon inflation and contraction thus can mechanically force the released therapeutic agent against and into the vessel wall.

Suitable piezoelectric coatings include without limitation poled poly(vinylidene fluoride). Piezoelectric coatings can additionally include graphitic carbon to improve coating conductivity.

In accordance with the subject matter disclosed above, encapsulation of the therapeutic agent can mitigate undesired effects associated with systemic release of the therapeutic agent during catheter delivery, and drug uptake into the vessel wall can be increased by the application of low voltage or current, e.g. via electroporation of the endothelium. Additionally, the encapsulant can be modified to provide moieties for ligand targeting to further improve drug delivery and retention.

In additional embodiments of the disclosed subject matter, the balloon can include microcapsules on its outer surface. In this regard, the microcapsules are configured to encompass the therapeutic agent. Upon inflation of the balloon the microcapsules located on the surface of the balloon contact the tissue of the arterial wall. Alternatively, the microcapsules can be formed in the wall of the balloon surface. The therapeutic agent can be released from the microcapsules by fracturing of the microcapsules and/or diffusion from the microcapsule into the arterial wall. The microcapsules can be fabricated in accordance with the methods disclosed in U.S. Pat. No. 5,1023,402 to Dror or U.S. Pat. No. 6,129,705 to Grantz and the patents referenced therein, each of which is incorporated herein by reference in its entirety. According to this aspect of the disclosed subject matter, the microcapsules can be configured to dissolve or fracture upon exposure to electric current or voltage. Additionally or alternatively, the microcapsules can be configured to fracture upon swelling of the coating and/or conductive polymer upon reduction/oxidation of the conductive polymer and concomitant solvent absorption and swelling.

In accordance another aspect of the disclosed subject matter, an outer fibrous coating can be electrospun or stretched onto the medical device or balloon catheter. During balloon inflation, the therapeutic formulation or coating is stretched and allows for coating solubilization and release. The fiber diameters and material properties can be fine tuned for optimal pore size and to release the particles containing the therapeutic agent. Fibrous coatings on expandable members are described in U.S. patent application Ser. No. 12/237,998 to R. von Oepen and U.S. patent application Ser. No. 12/238,026 to K. Ehrenreich, the disclosures of which are incorporated by reference in their entirety. Additionally or alternatively, the fiber coating can be composed of a conductive polymer, such as polyaniline. The fiber coating can be loaded with drug and configured to electophoretically repel drug and/or to reversibly oxidize and hydrate to permit elution of the therapeutic agent by diffusion.

Preferably, the coating exhibits sufficient flexibility and elasticity to retain its mechanical integrity upon balloon inflation and recover its initial configuration upon deflation of the balloon. One or more plasticizers can be incorporated into the balloon coating and/or the conductive polymer coating to improve its mechanical integrity on inflation and deflation. Plasticizers can improve the capacity for elongation of the conductive polymers disclosed herein, promoting mechanical integrity upon balloon inflation. Suitable plasticizers are low molecular weight, and water soluble species that are essentially non-volatile. The plasticizers include, for the purpose of illustration and without limitation, DMSO, polyethylene glycol (Molecular Weight<40K), propylene glycol, polypropylene glycol, glycerol, N-methyl-2-pyrrolidone (NMP), DMAC, benzyl alcohol, and fatty alcohols. Polyethylene glycol, polypropylene glycol, glycerin, and organic solvents are particularly suited to the applications disclosed herein.

In accordance with the disclosed subject matter, and for purpose of illustration and not limitation, the therapeutic agent or drug can antithrombotics, anticoagulants, antiplatelet agents, anti-lipid agents, thrombolytics, antiproliferatives, anti-inflammatories, agents that inhibit hyperplasia, smooth muscle cell inhibitors, antibiotics, growth factor inhibitors, cell adhesion inhibitors, cytostatic agents, cell adhesion promoters, antimitotics, antifibrins, antioxidants, antineoplastics, agents that promote endothelial cell recovery, antiallergic substances, viral vectors, nucleic acids, monoclonal antibodies, antisense compounds, oligonucleotides, cell permeation enhancers, radiopaque agent markers, HMG CoA reductase inhibitors, pro-drugs and combinations thereof.

The term “anti-proliferative” as used herein means an agent used to inhibit cell growth, such as chemotherapeutic drugs. Some non-limiting examples of anti-proliferative drugs include taxanes, paclitaxel, and protaxel. Anti-proliferative agents can be anti-mitotic. The term “anti-mitotic” as used herein means an agent used to inhibit or affect cell division, whereby processes normally involved in cell division do not take place. One sub-class of anti-mitotic agents includes vinca alkaloids. Representative examples of vinca alkaloids include, but are not limited to, vincristine, paclitaxel, etoposide, nocodazole, indirubin, and anthracycline derivatives, including, for example, daunorubicin, daunomycin, and plicamycin. Other sub-classes of anti-mitotic agents include anti-mitotic alkylating agents, including, for example, tauromustine, bofumustine, and fotemustine, and anti-mitotic metabolites, including, for example, methotrexate, fluorouracil, 5-bromodeoxyuridine, 6-azacytidine, and cytarabine. Anti-mitotic alkylating agents affect cell division by covalently modifying DNA, RNA, or proteins, thereby inhibiting DNA replication, RNA transcription, RNA translation, protein synthesis, or combinations of the foregoing. An example of an anti-mitotic agent includes, but is not limited to, paclitaxel. As used herein, paclitaxel includes the alkaloid itself and naturally occurring forms and derivatives thereof, as well as synthetic and semi-synthetic forms thereof.

Anti-platelet agents are therapeutic entities that act by (1) inhibiting adhesion of platelets to a surface, typically a thrombogenic surface, (2) inhibiting aggregation of platelets, (3) inhibiting activation of platelets, or (4) combinations of the foregoing. Activation of platelets is a process whereby platelets are converted from a quiescent, resting state to one in which platelets undergo a number of morphologic changes induced by contact with a thrombogenic surface. These changes include changes in the shape of the platelets, accompanied by the formation of pseudopods, binding to membrane receptors, and secretion of small molecules and proteins, including, for example, ADP and platelet factor 4. Anti-platelet agents that act as inhibitors of adhesion of platelets include, but are not limited to, eptifibatide, tirofiban, RGD (Arg-Gly-Asp)-based peptides that inhibit binding to gpIIbIIIa or avb3, antibodies that block binding to gpIIaIIIb or avb3, anti-P-selectin antibodies, anti-E-selectin antibodies, compounds that block P-selectin or E-selectin binding to their respective ligands, saratin, and anti-von Willebrand factor antibodies. Agents that inhibit ADP-mediated platelet aggregation include, but are not limited to, disagregin and cilostazol.

As discussed above, at least one therapeutic agent can be an anti-inflammatory agent. Non-limiting examples of anti-inflammatory agents include prednisone, dexamethasone, hydrocortisone, estradiol, triamcinolone, mometasone, fluticasone, clobetasol, and non-steroidal anti-inflammatories, including, for example, acetaminophen, ibuprofen, naproxen, adalimumab and sulindac. The arachidonate metabolite prostacyclin or prostacyclin analogs is an example of a vasoactive antiproliferative. Other examples of these agents include those that block cytokine activity or inhibit binding of cytokines or chemokines to the cognate receptors to inhibit pro-inflammatory signals transduced by the cytokines or the chemokines. Representative examples of these agents include, but are not limited to, anti-IL1, anti-IL2, anti-IL3, anti-IL4, anti-IL8, anti-IL15, anti-IL18, anti-MCP1, anti-CCR2, anti-GM-CSF, and anti-TNF antibodies.

Anti-thrombotic agents include chemical and biological entities that can intervene at any stage in the coagulation pathway. Examples of specific entities include, but are not limited to, small molecules that inhibit the activity of factor Xa. In addition, heparinoid-type agents that can inhibit both FXa and thrombin, either directly or indirectly, including, for example, heparin, heparin sulfate, low molecular weight heparins, including, for example, the compound having the trademark Clivarin®, and synthetic oligosaccharides, including, for example, the compound having the trademark Arixtra®. Also included are direct thrombin inhibitors, including, for example, melagatran, ximelagatran, argatroban, inogatran, and peptidomimetics of binding site of the Phe-Pro-Arg fibrinogen substrate for thrombin. Another class of anti-thrombotic agents that can be delivered is factor VII/VIIa inhibitors, including, for example, anti-factor VII/VIIa antibodies, rNAPc2, and tissue factor pathway inhibitor (TFPI).

Thrombolytic agents, which can be defined as agents that help degrade thrombi (clots), can also be used as adjunctive agents, because the action of lysing a clot helps to disperse platelets trapped within the fibrin matrix of a thrombus. Representative examples of thrombolytic agents include, but are not limited to, urokinase or recombinant urokinase, pro-urokinase or recombinant pro-urokinase, tissue plasminogen activator or its recombinant form, and streptokinase.

Furthermore, the therapeutic agents include a cytostatic agent. The term “cytostatic” as used herein means an agent that mitigates cell proliferation, allows cell migration, and does not induce cell toxicity. These cytostatic agents include, for the purpose of illustration and without limitation, macrolide antibiotics, zotarolimus, sirolimus, rapamycin, everolimus, biolimus, umirolimus, myolimus, novolimus, temsirolimus, deforolimus, ridaforolimus, tacrolimus, pimecrolimus, derivatives and analogues thereof, any macrolide immunosuppressive drugs, and combinations thereof. Other therapeutic agents include cytotoxic drugs, including, for example, apoptosis inducers, including TGF, and topoisomerase inhibitors, including, 10-hydroxycamptothecin, irinotecan, and doxorubicin.

A wide variety of balloon catheters and balloon constructs are known and suitable for use in accordance with the disclosed subject matter. For purpose of illustration and not limitation, the expandable member is fabricated from polymeric material such as compliant, non-compliant or semi-compliant polymeric material or polymeric blends (e.g., a mixture of polymers). In one embodiment, the polymeric material is compliant such as but not limited to a polyamide/polyether block copolymer (commonly referred to as PEBA or polyether-block-amide). In some embodiments, the polyamide and polyether segments of the block copolymers can be linked through amide or ester linkages. The polyamide block can be selected from various aliphatic or aromatic polyamides known in the art. In some embodiments, the polyamide is aliphatic. Some non-limiting examples include nylon 12, nylon 11, nylon 9, nylon 6, nylon 6/12, nylon 6/11, nylon 6/9, and nylon 6/6. In some embodiments, the polyamide is nylon 12. The polyether block can be selected from various polyethers known in the art. Some non-limiting examples of polyether segments include poly(tetramethylene ether), tetramethylene ether, polyethylene glycol, polypropylene glycol, poly(pentamethylene ether) and poly(hexamethylene ether). Commercially available PEBA material can also be utilized such as for example, PEBAX® materials supplied by Arkema (France). Various techniques for forming a balloon from polyamide/polyether block copolymer is known in the art. One such example is disclosed in U.S. Pat. No. 6,406,457 to Wang, the disclosure of which is incorporated by reference.

In other embodiments, the balloon material is formed from polyamides. In some embodiments, the polyamide has substantial tensile strength, be resistant to pin-holing even after folding and unfolding, and be generally scratch resistant, such as those disclosed in U.S. Pat. No. 6,500,148 to Pinchuk, the disclosure of which is incorporated herein by reference. Some non-limiting examples of polyamide materials suitable for the balloon include nylon 12, nylon 11, nylon 9, nylon 69 and nylon 66. In some embodiments, the polyamide is nylon 12. Other suitable materials for constructing non-compliant balloons are polyesters such as poly(ethylene terephthalate) (PET), Hytrel thermoplastic polyester, and polyethylene.

In another embodiment, the balloon is formed of a polyurethane material, such as TECOTHANE® (Thermedics). TECOTHANE® is a thermoplastic, aromatic, polyether polyurethane synthesized from methylene disocyanate (MDI), polytetramethylene ether glycol (PTMEG) and 1,4 butanediol chain extender. TECOTHANE® grade 1065D is one suitable embodiment, and has a Shore durometer of 65D, an elongation at break of about 300%, and a high tensile strength at yield of about 10,000 psi. However, other suitable grades can be used, including TECOTHANE® 1075D, having a Shore D hardness of 75. Other suitable compliant polymeric materials include ENGAGE® (DuPont Dow Elastomers (an ethylene alpha-olefin polymer) and EXACT® (Exxon Chemical), both of which are thermoplastic polymers. Other suitable compliant materials include, but are not limited to, elastomeric silicones, latexes, and urethanes.

The compliant material can be cross linked or uncrosslinked, depending upon the balloon material and characteristics required for a particular application. Some suitable polyurethane balloon materials are not crosslinked. However, other suitable materials, such as the polyolefinic polymers ENGAGE® and EXACT®, can be crosslinked. By crosslinking the balloon compliant material, the final inflated balloon size can be controlled. Conventional crosslinking techniques can be used including thermal treatment and E-beam exposure. After crosslinking, initial pressurization, inflation, and preshrinking, the balloon will thereafter expand in a controlled manner to a reproducible diameter in response to a given inflation pressure, and thereby avoid overexpanding the stent (if used in a stent delivery system) to an undesirably large diameter.

In further embodiments, the balloon is formed from a low tensile set polymer such as a silicone-polyurethane copolymer. In certain embodiments, the silicone-polyurethane is an ether urethane and more specifically an aliphatic ether urethane such as PURSIL AL 575A and PURSIL AL10, (Polymer Technology Group), and ELAST-EON 3-70A, (Elastomedics), which are silicone polyether urethane copolymers, and more specifically, aliphatic ether urethane cosiloxanes. In an alternative embodiment, the low tensile set polymer is a diene polymer. A variety of suitable diene polymers can be used such as but not limited to an isoprene such as an AB and ABA poly(styrene-block-isoprene), a neoprene, an AB and ABA poly(styrene-block-butadiene) such as styrene butadiene styrene (SBS) and styrene butadiene rubber (SBR), and 1,4-polybutadiene. In some embodiments, the diene polymer is an isoprene including isoprene copolymers and isoprene block copolymers such as poly(styrene-block-isoprene). One suitable isoprene is a styrene-isoprene-styrene block copolymer, such as Kraton 1161K available from Kraton, Inc. However, a variety of suitable isoprenes can be used including HT 200 available from Apex Medical, Kraton R 310 available from Kraton, and isoprene (i.e., 2-methyl-1,3-butadiene) available from Dupont Elastomers. Neoprene grades useful in the disclosed subject matter include HT 501 available from Apex Medical, and neoprene (i.e., polychloroprene) available from Dupont Elastomers, including Neoprene G, W, T and A types available from Dupont Elastomers.

In accordance with another aspect of the disclosed subject matter, the outer surface of the balloon is modified. In this regard, the balloon surface can include a textured surface, roughened surface, voids, spines, channels, dimples, pores, or microcapsules or a combination thereof, as will be described below.

In accordance with the disclosed subject matter, the balloon does not include a stent or is free of a stent. However, a stent can be mounted onto the coated balloon. The stent will not detrimentally affect coating integrity or drug delivery. The type of stent that can be used includes, but is not limited to, bare metal stent, balloon expandable stent, self expanding stent, drug eluting stent, prohealing stent, and self-expanding vulnerable plaque implant. The balloon can be coated independently of the stent or in conjunction with the stent coating process. The stent coating can contain the same or different therapeutic agents from the balloon catheter or expandable member. However, the particular coating on the balloon catheter or expandable member preferably has distinct release kinetics from the therapeutic coating on the stent.

In certain embodiments of the disclosed subject matter, the balloon is formed of a porous elastomeric material having at least one void formed in the wall of the balloon surface. For example, the entire cross section of the balloon can contain a plurality of voids. Alternatively, the plurality of void can be distributed along select lengths of the balloon outer surface. For example and not limitation, the plurality of voids can be distributed only along the working section of the balloon. The voids define an open space within the outer surface of the balloon. In some embodiments, the therapeutic agent is dispersed within the space defined by the plurality of voids across the cross section of the balloon outer surface.

In operation, the therapeutic agent is released or is expelled from the pores upon inflation of the balloon. In this regard, the durometer of the polymeric material of the balloon surface and in particular the depression of the void is sufficiently flexible to allow for expulsion of the therapeutic agent and/or coating contained within the plurality of voids upon inflation of the balloon. The expelled coating with therapeutic agent is released into the vessel lumen or into the tissue surrounding and contacting the inflated balloon.

In further embodiments, the balloon includes protrusions configured to contact or penetrate the arterial wall of a vessel upon inflation of the balloon. A therapeutic formulation is disposed on the protrusions and when inflated the therapeutic formulation and/or therapeutic agent coats or adheres to the tissue of the arterial wall. Alternatively, the balloon can include two concentric balloons in a nesting configuration. The therapeutic formulation is disposed between the two concentric balloons. Thus, the space between the two concentric balloons; one being an interior balloon and the other being an exterior balloon, acts as a reservoir. In this regard, the protrusions can include apertures for expulsion of the therapeutic formulation and/or therapeutic agent upon inflation of the interior and exterior concentric balloons. For example, as described in U.S. Pat. No. 6,991,617 to Hektner, the disclosure of which is incorporated herein by reference thereto. In another embodiment, the balloon can include longitudinal protrusions configured to form ridges on the balloon surface. As described in U.S. Pat. No. 7,273,417 to Wang, the entire disclosure of which is incorporated herein by reference, the ridges can be formed of filaments spaced equidistantly apart around the circumference of the balloon. However, a larger or smaller number of ridges can alternatively be used. The longitudinal ridges can be fully or partially enveloped by the polymeric material of the balloon.

In accordance with another aspect of the disclosed subject matter, if desired, a protective sheath can be utilized to protect the therapeutic formulation from being rubbed off of the balloon during the movement of the coated balloon through the body lumen. The sheath is made in certain embodiments from an elastic and resilient material which conforms to the shape of the balloon and in particular is capable of expanding upon inflation of the balloon. The sheath can include apertures along a length thereof. In operation, the inflation of the balloon causes the apertures of the sheath to widen for release of the therapeutic formulation and/or therapeutic agent to the tissue of the arterial wall. In some embodiments, the sheath has a thickness less than 10 mils. However, other thicknesses are possible.

In another embodiment, the sheath has at least one longitudinal line of weakness allowing the sheath to rupture upon inflation of the balloon and the release of the therapeutic formulation and/or therapeutic agent onto the tissue of the arterial wall of the vessel. In some embodiments, the sheath is formed from polymeric material known to be suitable for use in balloon catheters. In additional embodiments, the sheath material is an elastomeric material which will also spring back when it splits to expose more of the body lumen to the coating. The line of weakness could be provided by various techniques known in the art. However, one non-limiting examples include perforating the sheath material. In operation, the sheath is placed over the coated balloon while in the deflated state. When the coated balloon is inflated, the sheath is expanded to the extent that it exceeds its elastic limit at the line of weakness and bursts to expose and therefore release the therapeutic formulation and/or therapeutic agent to the tissue of the arterial wall or vessel lumen. For example, see U.S. Pat. No. 5,370,614 to Amundson, the entire disclosure of which is incorporated by reference.

The disclosed subject matter can be embodied in other specific forms without departing from its spirit or essential characteristics. The described embodiments are to be considered in all respects only as illustrative and not restrictive. Thus, it is intended that the disclosed subject matter include modifications and variations that are within the scope of the appended claims and their equivalents. All references recited herein are incorporated herein in their entirety by specific reference.

1. A catheter for intraluminal delivery of a therapeutic agent to a subject comprising:
an elongate shaft having a proximal end portion, a distal end portion, and an inflation lumen defined therebetween;
an expandable member coupled at the distal end portion of the elongate shaft, the expandable member having a proximal end, a distal end, an outer surface, and an interior chamber defined therein;
an electrode disposed proximate the outer surface of the expandable member;
a coating disposed on at least a portion of the outer surface, the coating including a therapeutic agent; and
a power source in electrical communication with the electrode, optionally a cathode.

2. The catheter of claim 1, wherein the electrode is a film of conductive material, optionally wherein the conductive material is disposed on the balloon as a coating which defines the electrode, further optionally wherein the conductive material is a metal material optionally selected from the group consisting of gold, platinum, platinum iridium, palladium, tantalum, silver and niobium, or the conductive material is a polymeric material optionally selected from the group comprising polypyrrole, polyacetylene derivatives, poly(phenylene sulfide), polythiopene, and poly(3,4-ethylenedioxythiopene) and optionally includes carbon particles or metallic particles.

3. The catheter of claim 1 or claim 2, wherein the therapeutic agent is selected from the group consisting of antithrombotics, anticoagulants, antiplatelet agents, anti-lipid agents, thrombolytics, antiproliferatives, anti-inflammatories, agents that inhibit hyperplasia, smooth muscle cell inhibitors, antibiotics, growth factor inhibitors, cell adhesion inhibitors, cytostatic agents, cell adhesion promoters, antimitotics, antifibrins, antioxidants, antineoplastics, agents that promote endothelial cell recovery, antiallergic substances, viral vectors, nucleic acids, antisense compounds, oligonucleotides, cell permeation enhancers, radiopaque agent markers, HMG CoA reductase inhibitors, pro-drugs and combinations thereof, optionally wherein the therapeutic agent is a cytostatic drug optionally selected from the group consisting of rapamycin, sirolimus, zotarolimus, everolimus, deforolimus, ridaforolimus, biolimus, umirolimus, and tacrolimus, or wherein the therapeutic agent is an antiproliferative drug selected from the group consisting of paclitaxel, protaxel and docetaxel.

4. The catheter of any of the preceding claims, wherein the coating includes an emulsifier, optionally wherein the emulsifier is an anionic surfactant optionally selected from the group consisting of selected from the group consisting of phosphatidylglycerols, phosphatic acids, lysophospholipids, saturated fatty acids and unsaturated fatty acids or a polyionic polymer selected from the group consisting of polycationic polymers and polyanionic polymers.

5. The catheter of claim 4, wherein the emulsifier is a surfactant, further wherein the surfactant is a phosphatidylglycerol selected from the group consisting of EPG, DMPG, DPPG, DSPG, and POPG, a phosphatic acid selected from the group consisting of DMPA, DPPA, and DSPA, a lysophopholipid is selected from the group consisting of Lysophosphatidic acid (LPA), lyso-phosphatidylcholine (LPC), and sphingosine-1-phosphate (S1P), or an anionic fatty acid is selected from the group consisting of phosphatidylethanolamine, purified 90% soya phosphatidylcholine (trade name LECIVA-S90), purified egg lecithin (trade name LIPOVA-E120), phosphatidylserine, phosphatidylinositol 4,5-bisphosphate and phosphatidylinositol 3,4,5-triphosphate.

6. The catheter of claim 5, wherein the therapeutic agent is encapsulated in micelles, liposomes, microspheres, or nanoparticles of the surfactant.

7. The catheter of claim 1, wherein the power source is a direct current power source external to the body of the patient or wherein the power source is an integrated component of the catheter optionally, optionally wherein the power source is a battery, optionally further wherein the power source includes a timer and/or a fast acting fuse, and optionally further wherein the power source is connected to the electrode by an insulated electrical lead which optionally engages the electrode at the proximal end of the expandable member.

8. The catheter of any of the preceding claims, wherein the electrode comprises at least one conductive polymer and further wherein the electrode is disposed as a coating configured to absorb ions and water during temporary application of current or voltage to the conductive polymer, optionally wherein the conductive polymer is doped, further optionally wherein the at least one conductive polymer is selected from the group comprising polypyrrole, polyacetylene derivatives, poly(phenylene sulfide), polythiopene, and poly(3,4-ethylenedioxythiopene), and further optionally wherein the therapeutic agent is disposed in the coating in a matrix arrangement.

9. The catheter of claim 8, wherein the conductive polymer comprises nanoparticles selected from the group consisting of polymeric, metallic, and therapeutic agent nanoparticles or combinations thereof and/or wherein the conductive polymer is combined as a composite with an additional conductive polymer optionally selected from the group consisting of poly(vinylidene) fluoride, poly(vinylidene fluoride-co-hexafluoropropylene), poly(ester-amide), and a polyester.

10. The catheter of any of the preceding claims, wherein the coating further comprises a plasticizer optionally selected from the group consisting of glycerin, polyethylene glycol, polypropylene glycol, propylene glycol, tweens, N-methylpyrrolidone, dimethyl sulfoxide, benzyl benzoate, ethyl benzoate, benzyl alcohol, and phenoxyethanol.

11. The catheter of any of the preceding claims, wherein the coating is disposed as a first layer comprising a first conductive polymer and a first therapeutic agent and a second layer comprising a second conductive polymer and a second therapeutic agent, optionally wherein the first conductive polymer is different from the second conductive polymer and further optionally wherein the first therapeutic agent is different from the second therapeutic agent.

12. A catheter for intraluminal delivery of a therapeutic agent to a space within a patient comprising:
an elongate shaft having a proximal end portion, a distal end portion, and an inflation lumen defined therebetween;
an expandable member coupled at the distal end portion of the elongate shaft, the expandable member having a proximal end, a distal end, an outer surface, and an interior chamber defined therein;
a coating disposed on at least a portion of the outer surface, the coating including a therapeutic agent and a conductive polymer, wherein the conductive polymer is piezoelectric.

13. The catheter of claim 12, wherein the conductive polymer is selected from the group comprising poled poly(vinylidene fluoride) and poled poly(vinylidene fluoride-trifluoroethylene), and optionally wherein the coating further comprises graphitic carbon.

14. The coating of claim 12 or claim 13, wherein the therapeutic agent is encapsulated in a complex that dissolves in electric current optionally selected from the group consisting of gold, silver, porous gold nanoparticles, porous silver nanoparticles, gold-coated poly(vinylidene fluoride) nanoparticles, and silver-coated poly(vinylidene fluoride) nanoparticles.
